(19) United States
(12) Patent Application Publication
(10) Pub. No.: US 2025/0128037 A1
(43) Pub. Date: Apr. 24, 2025

(54) MICRONEEDLE STRUCTURE COMPRISING SHAPE-MEMORY POLYMER, AND MANUFACTURING METHOD THEREFOR

(71) Applicant: TMD Lab Co. Ltd, Seoul (KR)

(72) Inventors: Song Hyun LEE, Seoul (KR); Kang Suk LEE, Seoul (KR); Se Won YI, Seoul (KR); Mi Lan KANG, Seoul (KR)

(21) Appl. No.: 18/834,545

(22) PCT Filed: Jan. 13, 2023
(86) PCT No.: PCT/KR2023/000666

§ 371 Date: Jul. 30, 2024

(30) Foreign Application Priority Data
Feb. 11, 2022 (KR) 10-2022-0018239

(51) Int. Cl. A61M 37/00

(52) U.S. Cl. CPC A61M 37/0015; A61M 2037/0023; A61M 2037/0053; A61M 2205/0238; A61M 2207/10

(57) ABSTRACT

A microneedle structure is disclosed. The microneedle structure according to one aspect of the present invention comprises microneedles to be inserted into the skin, and may comprise: microneedles comprising a shape-memory polymer so that a first shape can be changed to a second shape by a predetermined external stimulus according to the characteristics of the shape-memory polymer, and a support member having the microneedles formed on one surface thereof.

### TECHNICAL FIELD

The present invention relates to a microneedle structure including a shape-memory polymer and a method of manufacturing the same, and more particularly, to a microneedle structure capable of being easily removed from the skin using the characteristics of a shape-memory polymer, and a method of manufacturing the same.

### BACKGROUND ART

Methods for delivering a drug into the human body include oral administration systems and transdermal drug delivery systems. The oral administration systems have the advantage of easy drug intake, but have the disadvantages of drug decomposition in the gastrointestinal tract, loss due to hepatic metabolism, and difficulty in eliminating the drugs after administration.

The transdermal drug delivery systems have the advantage of being more effective than the oral administration system because they deliver drugs directly into the body by injection, but have the disadvantage of causing severe pain, patient resistance, and skin damage.

To overcome the drawbacks of existing oral administration systems and transdermal drug delivery systems, microneedles and microneedle structures having a length of hundreds of micrometers (μm) have been developed. The microneedles and microneedle structures have the advantage of being able to deliver drugs directly into the body while minimizing skin damage without causing any pain.

Conventional microneedles and microneedle structures are configured so that only the drug is dissolved and the microneedles are removed when the microneedles coated with a drug are injected into the body, both the drug and the microneedles are dissolved after the microneedles are injected into the body, or the drug is delivered using a hydrogel formulation and a hollow structure of microneedles having a hole at the end thereof.

However, research on conventional microneedles and microneedle structures has been conducted in consideration of the aspect of drug delivery, and no active research and development has been conducted in terms of their removal after drug delivery or during use, and thus there has a problem in that it is difficult to remove the microneedles and microneedle structures after use.

Therefore, there has been a need for the development of a microneedle structure capable of being stably fixed to the skin while the drug is introduced into the body and also being easily removed from the skin after drug delivery or in situations where removal is necessary, and a method of manufacturing the same.

### DISCLOSURE

### Technical Problem

The present invention is designed to solve the above problems, and thus it is an object of the present invention to provide a microneedle and microneedle structure capable of being stably fixed after the microneedles are inserted into the body, and a method of manufacturing the same.

It is another object of the present invention to provide a microneedle and microneedle structure whose shape can be deformed after being inserted into the skin, and a method of manufacturing the same.

It is still another object of the present invention to provide a microneedle and microneedle structure capable of being easily removed from the skin, and a method of manufacturing the same.

However, the technical objects to be achieved in the present invention are not limited to the above-described technical objects, and thus it should be understood by those skilled in the art to which the present invention pertains that technical objects which are not described in this specification will become apparent from the detailed description of the present invention.

### Technical Solution

According to one aspect of the present invention, there is provided a microneedle structure including microneedles to be inserted into the skin, wherein the microneedle structure includes microneedles including a shape-memory polymer and formed to be deformed from a first shape to a second shape by a predetermined external stimulus according to the characteristics of the shape-memory polymer; and a support member having the microneedles formed on one surface thereof.

In this case, the first shape may include a body portion having a cross section that becomes smaller toward one side thereof; and a head portion extending from the one side of the body portion and having a cross section larger than that of the one side of the body portion.

In this case, the head portion may have a cross-section that becomes smaller toward a front end thereof.

In this case, the second shape may be a conical shape.

In this case, the second shape may be formed flat on the one surface of the support member.

In this case, the microneedles may be formed to be inserted into the skin while having the first shape, to be deformed from the first shape to the second shape while being inserted into the skin, and to be removed from the skin while having the second shape.

In this case, an outer shell layer may be formed on outer surfaces of the microneedles, and the outer shell layer may include a pharmaceutical composition.

In this case, an injection port may be formed at the end of each of the microneedles, an injection tube having one side connected to the injection port may be formed in each of the microneedles, and the microneedle structure may further include a storage portion connected to the other side of the injection tube to accommodate the pharmaceutical composition.

In this case, the predetermined stimulus may include at least one of a temperature change or ultraviolet irradiation.

In this case, the shape-memory polymer may include at least one of a biodegradable shape-memory polymer or a biocompatible shape-memory polymer.

In this case, the biocompatible shape-memory polymer may include at least one of polycaprolactone with a crosslinked acrylated end group, network-structured polycaprolactone, a polycaprolactone blend with polyurethane, cellulose-grafted polycaprolactone, polycaprolactone-co-polysiloxane, multi-arm-structured polycaprolactone, multi-arm-structured poly(lactic acid), a poly(lactic acid) copolymer, and poly(lactic acid)-co-poly(ethylene glycol).

In this case, the predetermined stimulus may include a temperature change, and the microneedles may be gradually deformed as the temperature changes around the reference temperature, and may have a first shape at a first temperature less than or equal to the reference temperature and a second shape at a second temperature higher than or equal to the reference temperature.

In this case, the reference temperature may range from 28° C. to 42° C.

In this case, the microneedles may be present in plural numbers, and the plurality of microneedles may be regularly arranged at regular intervals.

In this case, the plurality of microneedles may include first and second microneedles, and the first shape of the first microneedle may be different from the first shape of the second microneedle.

According to another aspect of the present invention, there is provided a method of manufacturing a microneedle structure, wherein the microneedle structure includes microneedles including a shape-memory polymer and formed to be deformed from a first shape to a second shape by a predetermined external stimulus according to the characteristics of the shape-memory polymer: and a support member having the microneedles formed on one surface thereof, and the method includes molding microneedles having the second shape using the shape-memory polymer: adjusting the temperature so that the shape of the microneedles having the second shape can be changed: and molding the microneedles so that the microneedles whose temperature is adjusted have the first shape.

In this case, the molding of the second shape may include applying the shape-memory polymer on one surface of a mold for the second shape on which an intaglio for the second shape corresponding to the second shape is formed: and applying pressure so that a portion of the shape-memory polymer is introduced into the intaglio for the second shape.

In this case, the molding of the second shape may include forming a support member by allowing the remainder other than the portion of the shape-memory polymer to spread on the one surface of the mold for the second shape centered on the intaglio for the second shape as pressure is applied to the shape-memory polymer.

In this case, the molding of the second shape may include mixing a thermal crosslinking initiator with the shape-memory polymer; and applying heat to the shape-memory polymer.

In this case, the thermal crosslinking initiator may include at least one of potassium persulfate, ammonium persulfate, benzoyl peroxide, dilauroyl peroxide, dicumyl peroxide, hydrogen peroxide, and azobisisobutyronitrile.

In this case, the molding of the second shape may include mixing a photo-crosslinking initiator with the shape-memory polymer: and applying ultraviolet rays to the shape-memory polymer.

In this case, the photo-crosslinking initiator may include at least one of Darocur, Irgacure, lithium phenyl-2,4,6-trimethylbenzoylphosphinate (LAP) having a phenyl phosphine structure, diphenyl(2,4,6-trimethylbenzoyl)phosphine (TPO), and ethyl(2,4,6-trimethylbenzoyl)phenylphosphinate (TPO-L).

In this case, the molding of the first shape may include disposing the microneedles having the second shape in intaglios for the first shape formed on one surface of a mold for the first shape to correspond to the first shape: and pressing the microneedles into the intaglios for the first shape.

In this case, after the molding of the first shape, the microneedle structure manufacturing method may include cooling the microneedles to a low temperature to fix the first shapes of the microneedles, and then maintaining the cooled state.

In this case, after the molding of the first shape, the microneedle structure manufacturing method may include attaching the microneedles having the first shape to the support member.

In this case, the molding of the first shape may include disposing the microneedles having the second shape in the intaglios for the first shape formed on one surface of a mold for the first shape to correspond to the first shape: applying an adhesive member to one side of each of the microneedles: and pressing the microneedles into the intaglios for the first shape.

### Advantageous Effects

According to the above configuration, the microneedle and microneedle structure according to an embodiment of the present invention, and the method of manufacturing the same can provide microneedles having an arrowhead shape to prevent the microneedles from separating after being inserted into the skin, thereby allowing the microneedles to be stably fixed to the skin after being inserted into the skin.

Also, the microneedle and microneedle structure according to an embodiment of the present invention, and the method of manufacturing the same can provide microneedles, which include a shape-memory polymer whose shape can be deformed, thereby deforming the shape of the microneedles after the microneedles are inserted into the skin.

In addition, the microneedle and microneedle structure according to an embodiment of the present invention, and the method of manufacturing the same can provide microneedles whose shape can be changed into a second shape having a low binding force with the skin after the microneedles are inserted into the skin while having a first shape having a high binding force with the skin, thereby easily removing the microneedles from the skin.

The effects of the present invention are not limited to the effects described above, and it should be understood that the effects of the present invention include all effects that can be inferred from the configuration of the invention described in the detailed description of the present invention or the appended claims.

### DESCRIPTION OF DRAWINGS

FIG. 1 is a perspective view of a microneedle structure according to a first embodiment of the present invention. Here, FIG. 1(a) shows the microneedles having a first shape, and FIG. 1(b) shows the microneedles having a second shape.

FIG. 2 is a diagram showing a process of inserting microneedles into the skin by the microneedle structure according to the first embodiment of the present invention.

FIG. 3 is a diagram showing a process in which the microneedles are deformed from a first shape to a second shape by applying a predetermined stimulus to the microneedle structure after the microneedles are inserted into the skin by the microneedle structure according to the first embodiment of the present invention.

FIG. 4 is a diagram showing a process of removing microneedles from the skin after the microneedles are deformed into the second shape by the microneedle structure according to the first embodiment of the present invention.

FIG. 5 is an image of an experiment performed to confirm the fixability of microneedles having the first shape according to the first embodiment of the present invention. Here, FIG. 5(a) is an image showing the microneedles inserted into hydrogel that simulates skin tissue, and FIGS. 5(b) and 5(c) are images showing a process of lifting the microneedles to the outside of the hydrogel.

FIG. 6 is a perspective view of a microneedle structure according to a second embodiment of the present invention. Here, FIG. 6(a) shows the microneedles having a first shape, and FIG. 6(b) shows the microneedles having a second shape.

FIG. 7 is a perspective view of a microneedle structure according to a third embodiment of the present invention. Here, FIG. 7(a) shows the microneedles having a first shape, and FIG. 7(b) shows the microneedles having a second shape.

FIG. 8 is a flowchart of a method of manufacturing a microneedle structure according to an embodiment of the present invention.

FIGS. 9A to 9D are diagrams for describing a method of manufacturing a microneedle structure according to a first embodiment of the present invention.

FIGS. 10A to 10E are diagrams for describing a method of manufacturing a microneedle structure according to a second embodiment of the present invention.

FIGS. 11A to 11D are diagrams for describing a method of manufacturing a microneedle structure according to a third embodiment of the present invention.

FIG. 12 is an image showing a microneedle structure array manufactured by the method of manufacturing a microneedle structure according to the embodiment of the present invention. Here, FIG. 12(a) is an image of microneedle structures molded into a second shape by any one step of the manufacturing method according to this embodiment, FIG. 12(b) is an image of the microneedle structures before an external stimulus is applied, and FIG. 12(c) is an image of the microneedle structures whose shape is deformed after the external stimulus is applied.

### MODE FOR INVENTION

Hereinafter, embodiments of the present invention will be described in detail with reference to the accompanying drawings so as to be easily carried out by those skilled in the art to which the present invention pertains. The present invention may be embodied in many different forms and is not limited to the embodiments described herein. In order to clearly describe the present invention, parts that are not relevant to the description in the drawings are omitted, and the same reference numerals are added to the same or similar elements throughout the specification.

Terms and words used in this specification and the claims should not be interpreted as limited to commonly used meanings or meanings in dictionaries and should be interpreted with meanings and concepts which are consistent with the technological scope of the present invention based on the principle which the inventors have appropriately defined concepts of terms in order to describe their invention in the best way.

It should be understood which the terms “comprise,” “include,” or the like, when used herein, specify the presence of stated features, numbers, steps, operations, elements, components, or groups thereof in this specification but do not preclude the presence or addition of one or more other features, numbers, steps, operations, elements, components, or groups thereof.

Unless there are special circumstances, a case in which a component is disposed “in front of,” “behind,” “above,” or “under” another component includes not only a case in which the component is disposed directly “in front of,” “behind,” “above,” or “under” another component, but also a case in which still another component is interposed therebetween. Unless there are special circumstances, a case in which some components are connected to each other includes not only a case in which the components are directly connected to each other, but also a case in which the components are indirectly connected to each other.

Microneedles and microneedle structures according to an embodiment of the present invention and methods of manufacturing the same are related to inventions that may utilize the characteristics of a shape-memory polymer to enable microneedles to be deformed from a shape having a high binding force with the skin to a shape having a low binding force with the skin, thereby providing microneedles capable of delivering drugs into the body while the microneedles are stably fixed to the skin and also being easily removed from the skin.

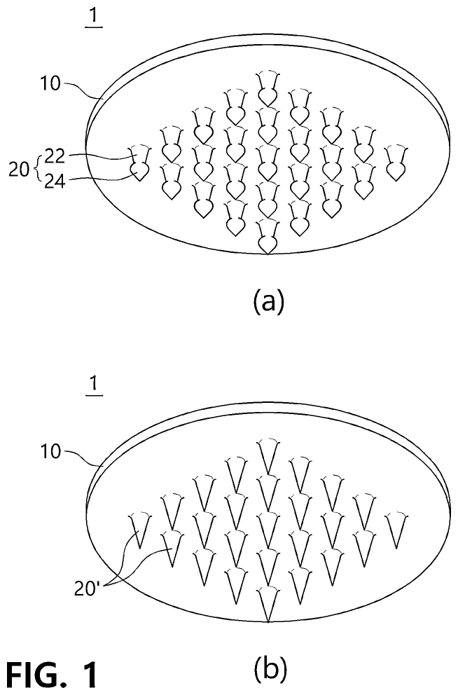

In describing the drawings below, each direction is defined and explained based on FIG. 1. More specifically, the direction in which microneedles protrude from a support member is defined as a downward direction, and the opposite direction is defined as an upward direction.

FIG. 1 is a perspective view of a microneedle structure according to a first embodiment of the present invention. Here, FIG. 1(a) shows the microneedles having a first shape, and FIG. 1(b) shows the microneedles having a second shape.

Referring to FIG. 1, the microneedle structure 1 according to the first embodiment of the present invention may include a support member 10 and microneedles 20 or 20′. The support member 10 may provide a base on which the microneedles 20 or 20′ are formed or attached.

The support member 10 may be made of a plate-shaped member having a predetermined thickness. At this time, the support member 10 may be formed to be easily anchored on the skin because the support member 10 has predetermined elasticity or flexibility so that the support member 10 can be easily bent. Of course, the support member 10 may also be made of a thin film.

The microneedles 20 or 20′ may be formed on one surface (i.e., a lower surface based on FIG. 1) of the support member 10. At this time, the microneedles 20 or 20′ may be present in plural numbers, and the plurality of microneedles 20 and 20′ may be regularly arranged to be spaced apart from each other at predetermined intervals.

In this embodiment, the plurality of microneedles 20 or 20′ may be arranged in 5rows and 5 columns on the lower surface of the support member 10 to have an overall square shape, but may be arranged in various ways depending on the characteristics of the body part to which the microneedles 20 or 20′ are inserted, the nature of a drug to be delivered, the dosage, and the time of administration. At this time, the support member may be made of a shape-memory polymer as will be described later.

Referring to FIG. 1(a), a microneedle 20 may have a first shape having a structure that has a high binding force with the skin so that the microneedle 20 can be stably fixed to the skin after being inserted into the skin.

In this case, the microneedle 20 having the first shape may be composed of a body portion 22 and a head portion 24. The body portion 22 may have a cross section that becomes smaller toward the outer side (i.e., a lower side based on FIG. 1) of the support member 10. The head portion 24 may be formed to extend from one side (i.e., a lower side based on FIG. 1) of the body portion 22, and may have a cross section larger than that of the one side of the body portion 22. At this time, the head portion 24 may have a cross section that becomes smaller toward a front end thereof.

In this case, an outer shell layer (not shown) including a pharmaceutical composition may be formed on the outer surface of the microneedle 20 having the first shape. Accordingly, after the microneedle 20 is inserted into the skin, the outer shell layer is dissolved by a body fluid and the like, thereby allowing the pharmaceutical composition to be delivered into the body.

Also, in order to deliver the pharmaceutical composition into the body, an injection port (not shown) may be formed at the end of the microneedle 20, and an injection pipe (not shown) having one side connected to the injection port may be formed in the microneedle 20. In this case, the microneedle structure may further include a storage portion (not shown) connected to the other side of the injection tube to accommodate the pharmaceutical composition.

Accordingly, after the microneedle 20 is inserted into the skin, the pharmaceutical composition accommodated in the storage portion may be introduced into the interior of the microneedle 20 through the injection tube, and then delivered into the body through the injection port.

Meanwhile, in this embodiment, the first shape of the microneedle 20 is composed of the body portion 22 and the head portion 24 as described above, but the first shape may be made of various shapes that have a high binding force with the skin so that the microneedle 20 can be stably fixed to the skin. For example, the first shape may be formed to have a hook-shaped end, a saw blade-shaped side, or an overall zigzag shape.

In addition, in this embodiment, the plurality of microneedles 20 have the same first shape, but if necessary, the plurality of microneedles 20 may each be configured to have different first shapes.

More specifically, the plurality of microneedles 20 may include first and second microneedles, and the first shapes of the first and second microneedles may be different from each other.

The microneedles 20 or 20′ according to the first embodiment of the present invention may include polycaprolactone-polyglycidyl methacrylate (PCL-PGMA) that is a shape-memory polymer.

Accordingly, the microneedles 20 or 20′ according to the first embodiment of the present invention may be deformed from the first shape to the second shape by a predetermined external stimulus according to the characteristics of the shape-memory polymer. At this time, the second shape may refer to a shape having a structure that has a low binding force with the skin so that the microneedle 20′ can be easily removed from the skin.

In this case, the shape-memory polymer may refer to a polymer that has a property of returning from a deformed shape to its previous shape by a predetermined external stimulus. As used in this specification, the term “biocompatibility” refers to a property of being substantially non-toxic to the human body and chemically inert and having no immunogenicity. As used in this specification, the term “biocompatible shape-memory polymer” refers to a polymer that has the properties according to the above-described “biocompatibility.”

As such, the microneedle structure 1 according to the first embodiment of the present invention may prevent negative effects on the human body because the microneedles 20 or 20′ are made of a biocompatible shape-memory polymer.

Meanwhile, in this embodiment, the shape-memory polymer includes a biocompatible shape-memory polymer, but the shape-memory polymer may include at least one of a biodegradable shape-memory polymer or a biocompatible shape-memory polymer.

In this specification, the term “biodegradability” refers to a property of being decomposed by body fluids or microorganisms in the living body, and the term “biodegradable shape-memory polymer” refers to a polymer that has the properties according to the above-described “biodegradability.”

The shape-memory polymer constituting the microneedles 20 or 20′ according to the first embodiment of the present invention is made of polycaprolactone-polyglycidyl methacrylate (PCL-PGMA), but the present invention is not limited thereto.

Specifically, the shape-memory polymer that may be used in the present invention may include at least one of segmented polyurethane, a polycaprolactone copolymer, (poly(lactic acid)), crosslinked polyethylene, crosslinked polycaprolactone, crosslinked poly(lactic acid), a crosslinked polyolefin, and crosslinked polysiloxane.

Also, the biocompatible or biodegradable shape-memory polymer that may be used in the present invention may include at least one of polycaprolactone with a crosslinked acrylated end group, network-structured polycaprolactone, a polycaprolactone blend with polyurethane, cellulose-grafted polycaprolactone, polycaprolactone-co-polysiloxane, multi-arm-structured polycaprolactone, multi-arm-structured poly(lactic acid), a poly(lactic acid) copolymer, and poly(lactic acid)-co-poly(ethylene glycol).

When the microneedles 20 or 20′ are made of a biodegradable shape-memory polymer, there is no need to remove the microneedles 20 or 20′ from the body after drug delivery. Even when the microneedles are not completely absorbed into the body and a residue remains, the microneedles 20 or 20′ may be deformed into a second shape having a low binding force with the skin, and thus may be easily removed from the skin.

Hereinafter, the second shape will be described first, and then the predetermined external stimulus will be described.

Referring to FIG. 1(b), the second shape of the microneedle 20′ may be a conical shape having a cross section that becomes smaller toward the outer side (i.e., a lower side based on FIG. 1) of the support member 10.

After the microneedle 20′ having the second shape is inserted into the skin, the elastic force of the skin acts outward from the skin. As a result, the microneedle 20′ having a low binding force to the skin may easily be removed from the skin.

In this embodiment, the second shape of the microneedle 20′ is a conical shape as described above, but the second shape may be made of various shapes that have a low binding force with the skin in order to easily detach the microneedle 20′ from the skin.

Referring again to FIGS. 1(a) and 1(b) together, the microneedles 20 or 20′ may be deformed from the first shape to the second shape by a predetermined external stimulus according to the characteristics of the shape-memory polymer because the microneedles 20 or 20′ includes a shape-memory polymer.

The predetermined external stimulus may be determined by the physical and chemical characteristics of the shape-memory polymer. In the first embodiment of the present invention, the external stimulus may include a temperature change. However, the external stimulus may include ultraviolet irradiation or transfer of energy, depending on the characteristics of the shape-memory polymer.

The temperature at which the microneedles 20 or 20′ are deformed from the first shape to the second shape is defined as the reference temperature. That is, the microneedles 20 or 20′ are gradually deformed as the temperature changes around the reference temperature, and may have a first shape at a first temperature less than or equal to the reference temperature and a second shape at a second temperature higher than or equal to the reference temperature.

In this case, the reference temperature may refer to a temperature at which deformation of the microneedles 20 or 20′ begins, but may also refer to a temperature at which deformation of the microneedles 20 or 20′ is noticeable or a temperature at which the microneedles 20 or 20′ begin to deform rapidly. That is, the microneedles 20 or 20′ may begin to deform even before the temperature of the microneedles 20 or 20′reaches the reference temperature.

The reference temperature may be determined by the characteristics of the shape-memory polymer constituting the microneedles 20 or 20′. For example, a user may set the reference temperature to 28° C. to 42° C. by appropriately selecting the shape-memory polymer.

Also, when thermal stimulation at a temperature much lower than the reference temperature is continuously applied for a long time, the shape of the microneedles 20 or 20′ may be gradually deformed depending on the characteristics of the shape-memory polymer constituting the microneedles 20 or 20′.

Hereinafter, a process of inserting microneedles into the skin and then removing the microneedles using the microneedle structure according to the first embodiment of the present invention will be described in detail.

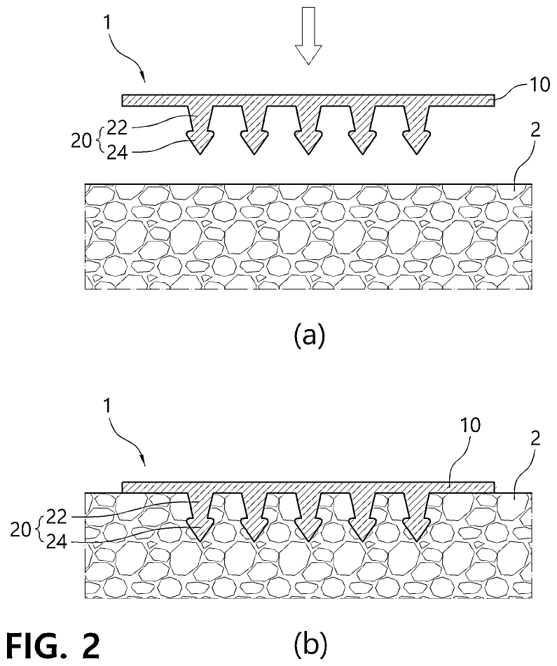

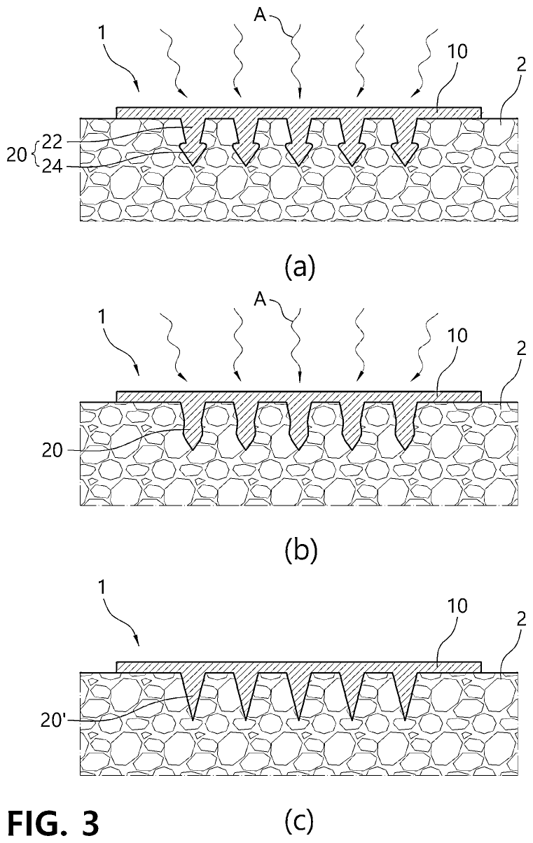

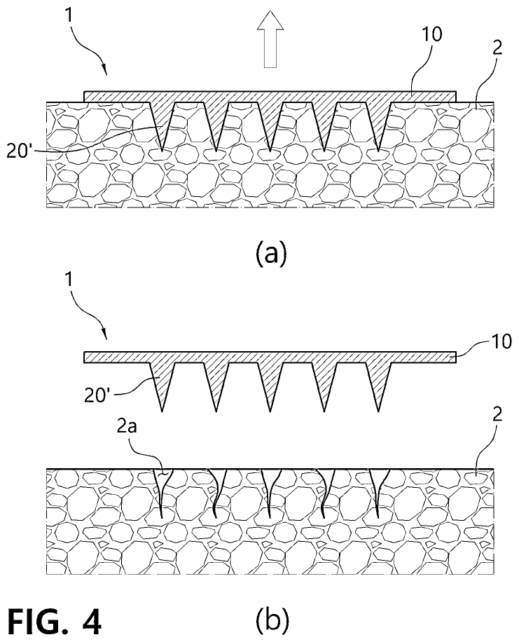

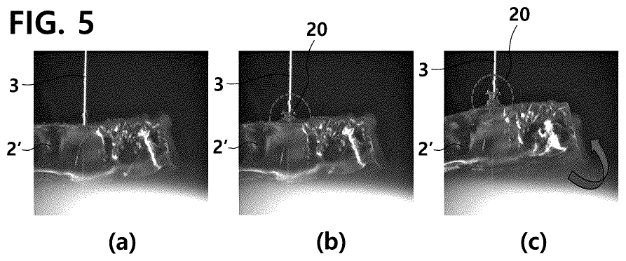

FIG. 2 is a diagram showing a process of inserting microneedles into the skin by the microneedle structure according to the first embodiment of the present invention. FIG. 3 is a diagram showing a process in which the microneedles are deformed from a first shape to a second shape by applying a predetermined stimulus to the microneedle structure after the microneedles are inserted into the skin by the microneedle structure according to the first embodiment of the present invention. FIG. 4 is a diagram showing a process of removing microneedles from the skin after the microneedles are deformed into the second shape by the microneedle structure according to the first embodiment of the present invention. FIG. 5 is an image of an experiment performed to confirm the fixability of microneedles having the first shape according to the first embodiment of the present invention. Here, FIG. 5(a) is an image showing the microneedles inserted into hydrogel that simulates skin tissue, and FIGS. 5(b) and 5(c) are images showing a process of lifting the microneedles to the outside of the hydrogel using a micropillar.

The microneedles according to the first embodiment of the present invention may be formed to be inserted into the skin while having a first shape having a high binding force with the skin, and to be removed from the skin while having a second shape having a low binding force with the skin.

Referring to FIG. 2(a), the microneedles 20 of the microneedle structure 1 according to the first embodiment of the present invention may be inserted into the skin 2 while having a first shape. For this purpose, a force that presses the microneedle structure 1 downward may be applied to an upper surface of the support member 10.

Referring to FIG. 2(b), as the microneedles 20 of the microneedle structure 1 according to the first embodiment of the present invention receive the downward force, the end of the head portion 24 may be inserted into the skin while tearing the tissue of the skin 2.

In this case, the tissue of the skin 2 torn by the head portion 24 may come into close contact with an outer peripheral surface of the body portion 22, which has a smaller cross section than the head portion 24, due to the elasticity of the skin 2. Accordingly, the upper side of the head portion 24 may be stably fixed by being caught by the tissue of the skin 2.

Referring to FIGS. 2 and 5(a), the microneedle 20 having the first shape according to the first embodiment of the present invention tears the hydrogel 2′ with a sharp end thereof so that the microneedle 20 can be easily inserted into the hydrogel 2′ even though the microneedle 20 is not pressed with a great force. Thereafter, the internal tissue of the hydrogel 2′ adheres to the microneedle 20 while surrounding the body portion 22 of the microneedle 20 due to the elasticity of the hydrogel 2′.

Referring to FIGS. 5(b) and 5(c), the microneedle 20 inserted into the hydrogel 2′ is pulled to the outside of the hydrogel 2′, but the head portion 24 of the microneedle 20 is caught in the internal tissue of the hydrogel 2′ surrounding the body portion 22 and does not come out.

These results show that the microneedle 20 according to the first embodiment of the present invention may be easily inserted into the skin 2 and may be fixed to the skin 2 after insertion so that a drug can be stably delivered into the body for a period of time desired by a user.

Referring to FIG. 3(a), the microneedle structure 1 according to the first embodiment of the present invention may receive an external stimulus A that can induce deformation of the shape-memory polymer from the outside.

The time at which the external stimulus A is applied may be selected by the user. For example, the time at which the external stimulus A is applied may be the time at which it is judged that the drug has been sufficiently administered into the skin 2 through the microneedle 20.

In this case, as long as the deformation of the shape of the microneedle 20 may be induced, there is no particular limitation on the type and number of external stimuli A applied to the microneedle 20, and the time and method for applying the stimulus.

For example, the external stimulus A may be applied using various methods (such as heat conduction through hot air, hot water, or a predetermined medium) applied to the microneedle structure 1 in order to reach the reference temperature, a temperature close to the reference temperature, or a temperature that is lower than the reference temperature but at which deformation of the microneedle 20 begins.

Referring to FIGS. 3(b) and 3(c), as the external stimulus A is applied to the microneedle structure 1, the microneedles 20 or 20′ may be deformed from the first shape to the second shape. In this case, the external stimulus A may be continuously applied. Also, the external stimulus A may be applied once or may be applied intermittently several times.

Referring to FIG. 4(a), in the microneedle structure 1 according to the first embodiment of the present invention, an external force may be applied to remove the microneedle structure 1 from the skin 2 after the microneedle 20′ is deformed into the second shape. Referring to FIG. 4(b), because the second shape has a conical shape having a cross section that becomes smaller toward a lower side thereof, the microneedle 20′ may be easily detached from the skin 2 due to the low binding force with the skin 2.

More specifically, because the skin 2 pushes the microneedle 20′ in a direction perpendicular to the outer peripheral surface of the microneedle 20′ in order to restore a portion 2a torn by the microneedle 20′, the microneedle 20′ having the second shape may be more easily pushed outward by the elastic force of the skin 2.

In this way, because the microneedles 20 or 20′ are inserted into the skin 2 in a state in which the microneedles 20 or 20′ have a first shape having a high bonding force with the skin 2, the microneedle structure 1 according to the first embodiment of the present invention may be stably fixed to the skin 2 to deliver drugs. Also, because the microneedles 20 or 20′ are detached from the skin 2 in a state in which the microneedles 20 or 20′ has a second shape having a low binding force with the skin 2, the microneedles 20 or 20′ may be easily removed from the skin 2.

Also, regarding the microneedle structure 1 according to the first embodiment of the present invention the reference temperature at which the deformation begins may be controlled by appropriately selecting the shape-memory polymer, and the timing of applying the temperature stimulus to reach the reference temperature may be controlled, which makes it possible to determine or control the timing of removing the microneedles 20 and 20′ from the skin 2.

Hereinafter, the microneedle structures according to the second and third embodiments of the present invention will be described. In this case, other configurations other than the second shapes of the support members and the microneedles of the microneedle structures according to the second and third embodiments of the present invention may be the same as those in the first embodiment, and thus a detailed description thereof will be omitted. Here, the second shapes of the support members and microneedles according to the second and third embodiments of the present invention will be described.

FIG. 6 is a perspective view of a microneedle structure according to a second embodiment of the present invention. Here, FIG. 6(a) shows the microneedles having a first shape, and FIG. 6(b) shows the microneedles having a second shape.

Referring to FIG. 6, a support member 110 of the microneedle structure 101 according to the second embodiment of the present invention may be made of a material other than the shape-memory polymer. For example, the support member 110 may be made using any known material such as medical tape, adhesive tape, or other types of polymers.

Also, a material constituting the support member 110 may be selected according to the physicochemical properties of the shape-memory polymer constituting the microneedles 120 or 120′. For example, when the shape-memory polymer deforms due to thermal stimuli, the support member 110 may be selected from a material having high heat transfer efficiency and configured to induce rapid deformation of the microneedles 120 or 120′. On the contrary, the support member 110 may be selected from a material having low heat transfer efficiency and configured to induce slow deformation of the microneedles 120 or 120′.

As such, regarding the microneedle structure 101 according to the second embodiment of the present invention, the material constituting the support member 110 may be appropriately selected depending on the body part and environment to which the microneedles 120 or 120′ are applied, thereby maximizing an effect of stably delivering drugs into the body and simultaneously removing the microneedles 120 or 120′ from the skin.

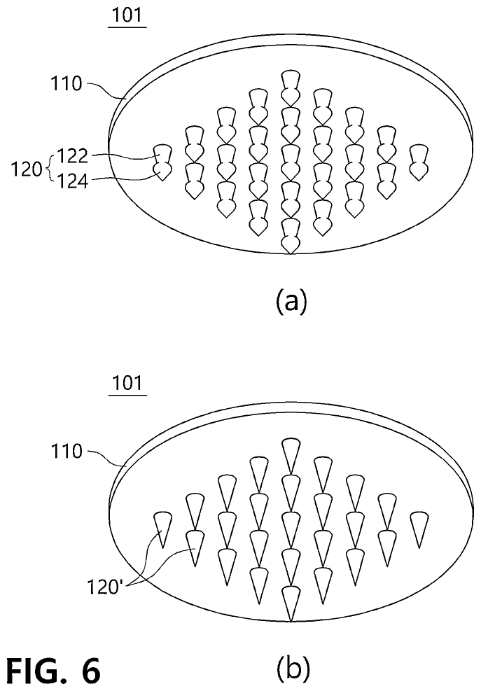

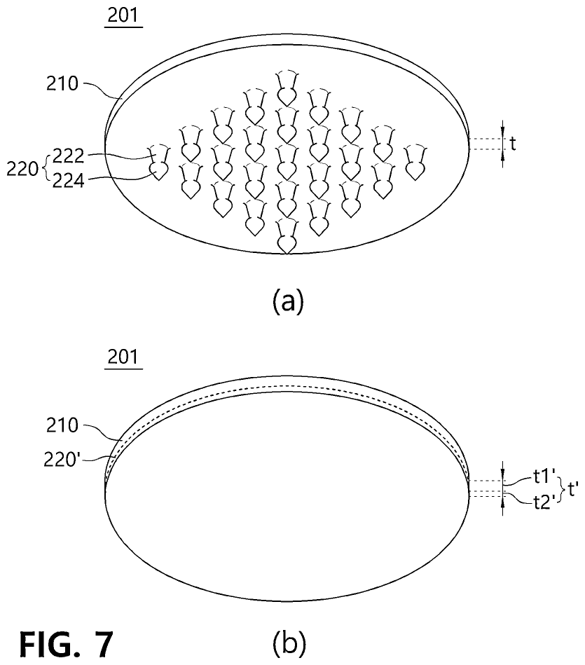

FIG. 7 is a perspective view of a microneedle structure according to a third embodiment of the present invention. Here, FIG. 7(a) shows the microneedles having a first shape, and FIG. 7(b) shows the microneedles having a second shape.

Referring to FIG. 7(a), the microneedle structure 201 according to the third embodiment of the present invention may be formed in the same manner as in the first embodiment in a state in which the microneedles 220 have the first shape.

Referring to FIG. 7(b), the second shape of the microneedles 220′ of the microneedle structure 201 according to the third embodiment of the present invention is formed flat and parallel to one surface of a support member 210.

In this case, the thickness t′ of the microneedle structure 201 in the state in which the microneedles 220′ have the second shape may be thicker than the thickness t of the support member 210 in the state in which the microneedles 220 have the first shape. More specifically, the thickness t′ of the microneedle structure 201 may be composed of the thickness t1′ of the support member 210 and the thickness t2′ of the second shape of the microneedles 220′.

In this case, the fact that the second shape is formed flat and parallel to one surface of the support member 210 means that even when the second shape does not protrude from the lower side of the support member 210 or has a slightly protruding shape, the protruding portion has a shape that may be easily removed from the skin, including a plane parallel to the outer surface of the skin into which the microneedles 220 or 220′ are inserted.

Also, the second shape of the microneedles 220′ has a shape that slightly protrudes from one side of the support member 210. In this case, because the second shape may be formed to have a curved surface having a very large curvature so that the protruding portion cannot be easily inserted into the skin, the microneedles 220′ may also have a shape that may be easily detached due to the low binding force with the skin.

Accordingly, when the second shape of the microneedles 220′ includes a plane facing the skin, the microneedle structure 201 according to the third embodiment of the present invention may be formed to be spontaneously detached from the skin in a process of deforming the microneedles 220 or 220′ from the first shape to the second shape.

Therefore, the microneedle structure 201 according to the third embodiment of the present invention may be easily removed from the skin even when no external force is applied to remove the microneedles 220 or 220′ from the skin.

Hereinafter, a method of manufacturing a microneedle structure according to an embodiment of the present invention will be described.

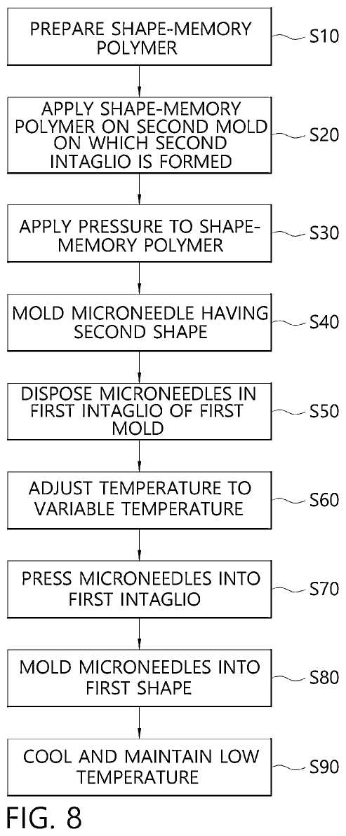

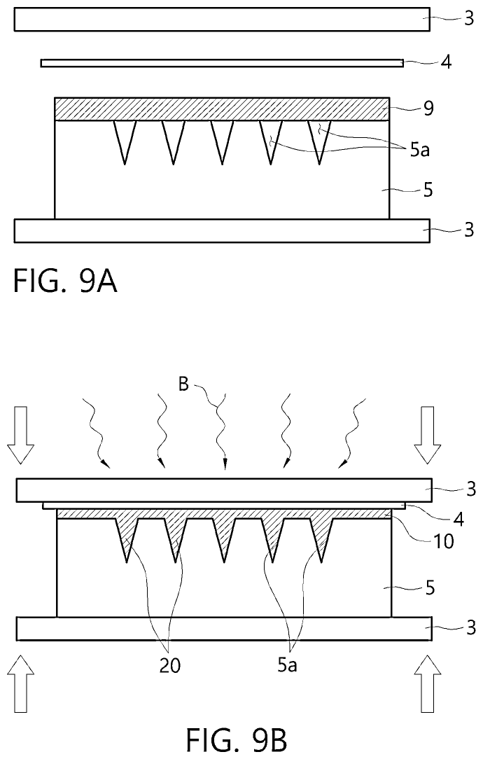

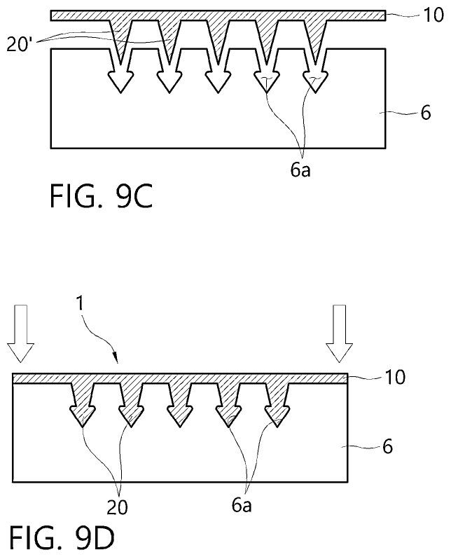

FIG. 8 is a flowchart of a method of manufacturing a microneedle structure according to an embodiment of the present invention. FIGS. 9A to 9D are diagrams for describing a method of manufacturing a microneedle structure according to the first embodiment of the present invention.

Referring to FIGS. 8 and 9A, the method of manufacturing a microneedle structure according to the first embodiment of the present invention is a method for manufacturing the microneedle structure according to the first embodiment of the present invention, and includes preparing a shape-memory polymer 9 (S10), and applying the prepared shape-memory polymer 9 to a mold 5 for the second shape (S20).

In this case, the shape-memory polymer 9 may be made of polycaprolactone-polyglycidyl methacrylate (PCL-PGMA), which is as a copolymer of caprolactone and glycidyl methacrylate.

Referring to FIG. 9A, an intaglio 5a for the second shape corresponding to the second shape may be formed on an upper surface of the mold 5 for the second shape. At this time, a plurality of intaglios 5a for the second shape may be formed on the upper surface of the mold 5 for the second shape to correspond to an array of a plurality of microneedles.

The mold 5 for the second shape may be made of various materials. For example, the mold 5 for the second shape may be made of polydimethylsiloxane (PDMS) as an ultraviolet-curable resin, but the present invention is not limited thereto.

Referring again to FIG. 9A, in the method of manufacturing a microneedle structure according to the first embodiment of the present invention, after the shape-memory polymer 9 is applied on the mold 5 for the second shape (S20), an intermediate member 4 may be disposed on the upper side of the shape-memory polymer 9 so that the upper side of the shape-memory polymer 9 can be pressed flat while receiving heat.

In this case, the intermediate member 4 may be made of a plate-shaped member having a predetermined thickness. The intermediate member 4 is preferably made of a material capable of heat transfer and capable of withstanding high pressure. For example, the intermediate member 4 may be made of glass, but the present invention is not limited thereto.

Referring to FIGS. 8, 9A, and 9B, in the method of manufacturing a microneedle structure according to the first embodiment of the present invention, after the intermediate member 4 is disposed on the upper side of the shape-memory polymer 9, pressure is applied to the shape-memory polymer 9 (S30), and the microneedle 20′ is molded into a second shape (S40).

In this case, a predetermined stimulus B may be applied along with pressure. The predetermined stimulus B may be determined by a process of molding the second shape of the shape-memory polymer 9. In this embodiment, the process of molding the second shape is a thermal crosslinking process, and the predetermined stimulus B includes heat transfer.

For this purpose, a thermal crosslinking initiator may be mixed with the shape-memory polymer 9. For example, at least one of potassium persulfate, ammonium persulfate, benzoyl peroxide, dilauroyl peroxide, dicumyl peroxide, hydrogen peroxide, and azobisisobutyronitrile may be mixed with the shape-memory polymer 9, but the present invention is not limited thereto. In this case, various known thermal crosslinking initiators may be used.

In this case, an amount of the thermal crosslinking initiator mixed into the shape-memory polymer 9 may have a weight ratio of 1% to 5% compared to the weight ratio of polycaprolactone-polyglycidyl methacrylate.

Meanwhile, as previously described in the microneedle structure according to the first embodiment of the present invention, the shape-memory polymer of the present invention is not limited to the polycaprolactone-polyglycidyl methacrylate, and may include at least one of various known shape-memory polymers, biocompatible shape-memory polymers, and biodegradable shape-memory polymers.

Referring again to FIG. 9B, the shape-memory polymer 9 may be heated and pressed for a predetermined time by a hot press plate 3. That is, a predetermined stimulus B may be applied by the hot press plate 3. At this time, the hot press plate 3 preferably heats both sides of the shape-memory polymer 9.

More specifically, hot press plates 3 may be disposed on the lower side of the mold 5 for the second shape and the upper side of the intermediate member 4, and then may heat and press the shape-memory polymer 9 by applying pressure in a direction that approaches each other.

In this case, the hot press plate 3 may perform a process of heating the shape-memory polymer 9 to 80° C. to 120° C. for approximately 15 minutes and applying a pressure of 5 MPa to 20 MPa to the shape-memory polymer 9. More preferably, the hot press plate 3 may perform a process of heating the shape-memory polymer 9 to 90° C. to 110° C. and applying a pressure of 13 MPa to 17 MPa to the shape-memory polymer 9.

Next, the microneedle 20′ is molded into a second shape (S40). Referring again to FIG. 9B, a portion of the shape-memory polymer 9 may be press-fitted into an intaglio 5a for the second shape of the mold 5 for the second shape as the portion of the shape-memory polymer 9 is pressed by the hot press plate 3. The remainder of the shape-memory polymer 9 may be spread on the upper surface of the mold 5 for the second shape centered on the intaglio 5a for the second shape to form a support member 10 having a predetermined thickness.

At the same time, the shape-memory polymer 9 may undergo a thermal crosslinking reaction by a predetermined stimulus B applied from the outside. As a result, the support member 10 may be formed, and microneedles 20′ having a second shape may be formed on one surface of the support member 10.

Thereafter, a process of removing the thermal crosslinking initiator mixed into the shape-memory polymer 9 may be further performed. At this time, the thermal crosslinking initiator mixed into the shape-memory polymer 9 may be removed by washing with water or blowing air.

Meanwhile, in this embodiment, the microneedles 20′ are molded through a thermal crosslinking reaction, but they may be molded through other reactions other than the thermal crosslinking reaction. For example, the microneedles 20′ may be molded through a photo-crosslinking reaction.

In this case, a photo-crosslinking initiator may be mixed with the shape-memory polymer 9. For example, at least one of Darocur having a hydroxy methylpropiophenone structure, Irgacure, lithium phenyl-2,4,6-trimethylbenzoylphosphinate (LAP) having a phenyl phosphine structure, diphenyl(2,4,6-trimethylbenzoyl)phosphine (TPO), and ethyl(2,4,6-trimethylbenzoyl)phenylphosphinate (TPO-L) may be mixed with the shape-memory polymer 9, but the present invention is not limited thereto.

In this case, the predetermined stimulus B may be ultraviolet rays, and the intermediate member 4 may be made of a material such as glass that may transmit ultraviolet rays. The predetermined stimulus B may be composed of ultraviolet rays having a wavelength of approximately 365 nm and emitted using a UV lamp, and the ultraviolet rays may be irradiated for approximately 200 seconds.

As described above, the method of manufacturing a microneedle structure according to the embodiment of the present invention may use a thermal crosslinking reaction or a photo-crosslinking reaction to mold the microneedle 20′ into the second shape, thereby reducing the reference temperature at which the microneedle 20′ is molded into the second shape.

Specifically, when the microneedle 20′ is molded without using a thermal crosslinking or photo-crosslinking reaction, the reference temperature may be set to approximately 30° C. to 48° C. On the other hand, when the microneedle 20′ is molded using a thermal crosslinking or photo-crosslinking reaction, the standard temperature may be reduced to approximately 28° C. to 42° C.

Accordingly, as the method of manufacturing a microneedle structure according to the embodiment of the present invention may use a thermal crosslinking or photo-crosslinking reaction to mold the microneedle 20′ into the second shape, the reference temperature at which the microneedle 20′ is deformed from the first shape to the second shape can be set to suit the body.

Referring again to FIGS. 8 and 9C, in the method of manufacturing a microneedle structure according to the first embodiment of the present invention, after the second shape is molded (S40), the microneedles 20′ are disposed inside intaglios 6a for the first shape of a mold 6 of the first shape (S50). Thereafter, the temperature of the microneedle 20′ is adjusted to a variable temperature (S60).

More specifically, an intaglio 6a for the first shape corresponding to the first shape may be formed on an upper surface of the mold 6 for the first shape. At this time, the intaglio 6a for the first shape may be formed in plural numbers corresponding to an array of a plurality of microneedles 20′.

The mold 6 for the first shape may be made of various materials. For example, the mold 6 for the first shape may be made of polydimethylsiloxane as an ultraviolet curable resin, but the present invention is not limited thereto.

The microneedles 20′ disposed in the intaglios 6a for the first shape of the mold 6 for the first shape may undergo a heat transfer process to have a variable temperature. At this time, the variable temperature may refer to a temperature at which the microneedles 20′ molded into the second shape may be molded into different shapes. In this embodiment, the variable temperature may be a temperature of 42° C. or higher.

Referring to FIGS. 8 and 9D, in the method of manufacturing a microneedle structure according to the first embodiment of the present invention, after the temperature is adjusted to a variable temperature (S60), the microneedle 20′ is pressed into the intaglio 6a for the first shape (S70) and molded into the first shape (S80). That is, the microneedle 20′ may be molded from the second shape to the first shape as the microneedle 20′ is pressed into the intaglio 6a for the first shape by an external force.

Next, in the method of manufacturing a microneedle structure according to the first embodiment of the present invention, after the microneedle structure 1 is cooled, the microneedle structure 1 is maintained at a low temperature for a predetermined time (S90) to fix the shape of the microneedles 20 to the first shape.

In this case, the cooling process may be a rapid cooling process, and the low temperature state may be achieved by maintaining the microneedle structure 1 below 0° C. The cooling process and the low temperature maintenance process may be performed using a cooler, ice, or liquid nitrogen.

In this way, the method of manufacturing a microneedle structure according to the first embodiment of the present invention may manufacture the microneedle structure 1 according to the first embodiment of the present invention.

Hereinafter, the methods for manufacturing a microneedle structure according to the second and third embodiments of the present invention will be described. In this case, the description of the same content as the first embodiment of the present invention will be omitted, and the content different from the first embodiment will be described.

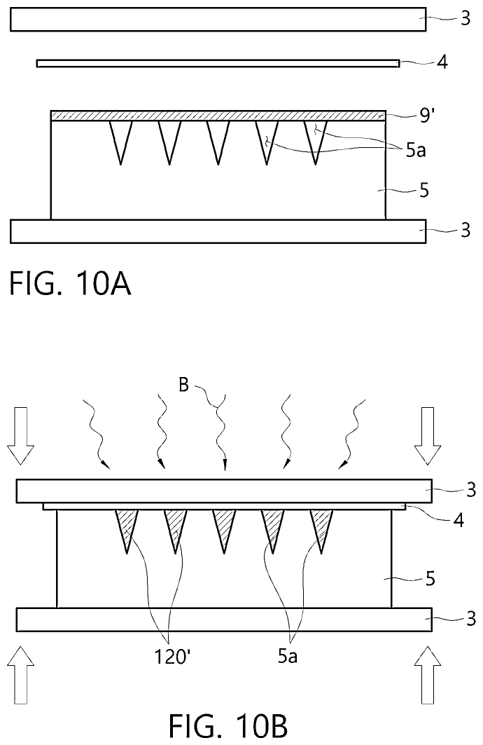

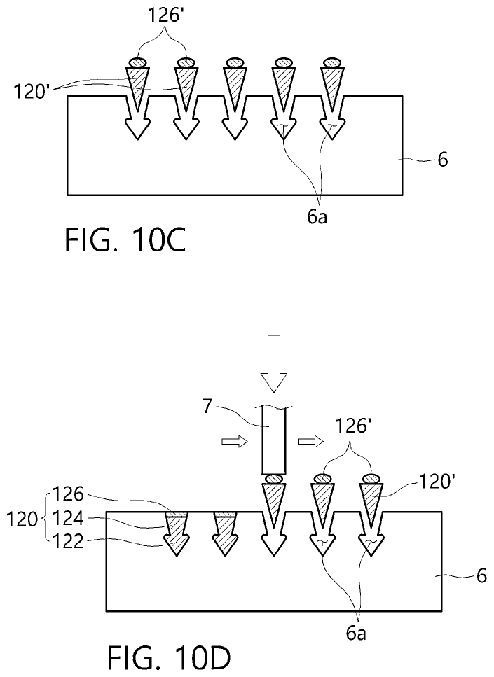

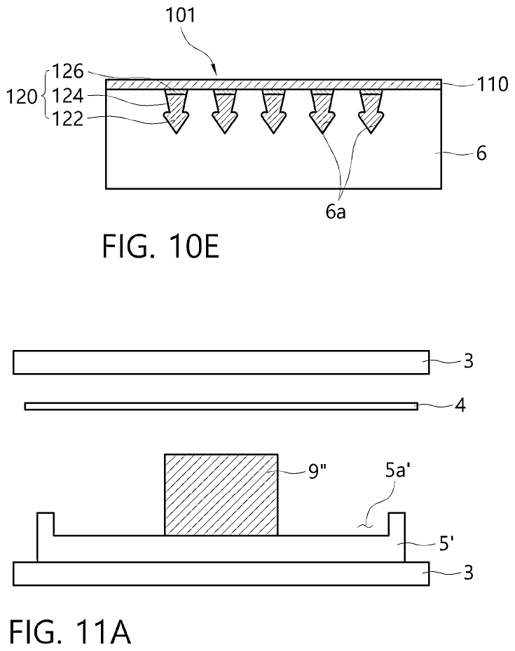

FIGS. 10A to 10E are diagrams for describing a method of manufacturing a microneedle structure according to the second embodiment of the present invention.

Referring to FIGS. 8, 10A, and 10B, the method of manufacturing a microneedle structure according to the second embodiment includes applying the shape-memory polymer 9′ on the mold 5 for the second shape (S10, S20), and disposing the intermediate member 4 on the upper side of the applied shape-memory polymer 9′ and pressing the shape-memory polymer 9′ using the hot press plate 3 (S40). As a result, the shape-memory polymer 9′ is press-fitted into the intaglio 5a for the second shape.

Next, a predetermined stimulus B is applied to the shape-memory polymer 9′ to cause a crosslinking reaction, thereby molding the microneedles 120′ into a second shape (S50). In this case, when the crosslinking reaction is a thermal crosslinking reaction, the predetermined stimulus B may be heat transfer by the hot press plate 3. On the other hand, when the crosslinking reaction is a photo-crosslinking reaction, the predetermined stimulus B may be composed of ultraviolet rays irradiated by a UV lamp. At this time, the shape-memory polymer 9′ may be press-fitted into the intaglio 5a for the second shape so that the plurality of microneedles 120′ separated from each other can be molded into the second shape.

Referring to FIG. 10C, the plurality of microneedles 120′ molded into the second shape are respectively disposed inside the plurality of intaglios 6a for the first shape formed on one surface of the mold 6 for the first shape (S50). At this time, an adhesive member 126′ may be placed on the upper side of the microneedle 120′.

In this case, the adhesive member 126′ may be made of various materials having an adhesive strength capable of attaching the microneedles 120′ to a support member, which will be described later. In this embodiment, the adhesive member 126′ may be made of photoresist epoxy that may be cured by ultraviolet rays to have adhesive strength, but the present invention is not limited thereto. For example, the adhesive member 126′ may be made of an industrial bonding or medical adhesive.

Referring to FIGS. 8 and 10D, in the method of manufacturing a microneedle structure according to the second embodiment of the present invention, after the temperature of the microneedles 120′ is adjusted to a variable temperature (S60), a micropillar 7 serves to press the microneedles 120′ into the intaglios 6a for the first shape (S70), and the microneedles 120′ are molded into the first shape (S80).

Then, referring to FIG. 10E, in the second embodiment of the present invention, after the support member 110 is placed on the upper sides of the microneedles 120, a process of attaching the microneedles 120 to the support member 110 is performed.

In this case, the support member 110 may be made of a material different from the material constituting the microneedles 120. For example, the support member 110 may be medical tape, general adhesive tape, or a member made of various polymers.

Then, the support member 110 and the microneedles 120 molded into the first shape are cooled, and then maintained at a low temperature (S90), thereby fixing the shape of the microneedles 120 to the first shape.

As described above, the method of manufacturing a microneedle structure according to the second embodiment of the present invention may manufacture the microneedle structure 101 according to the second embodiment of the present invention.

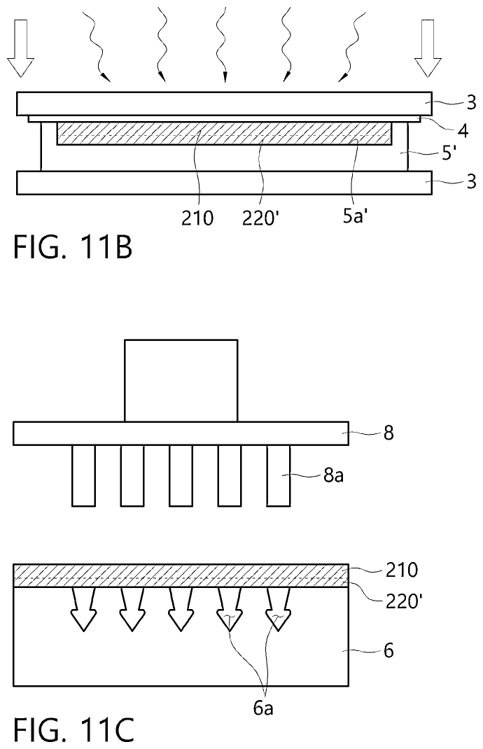

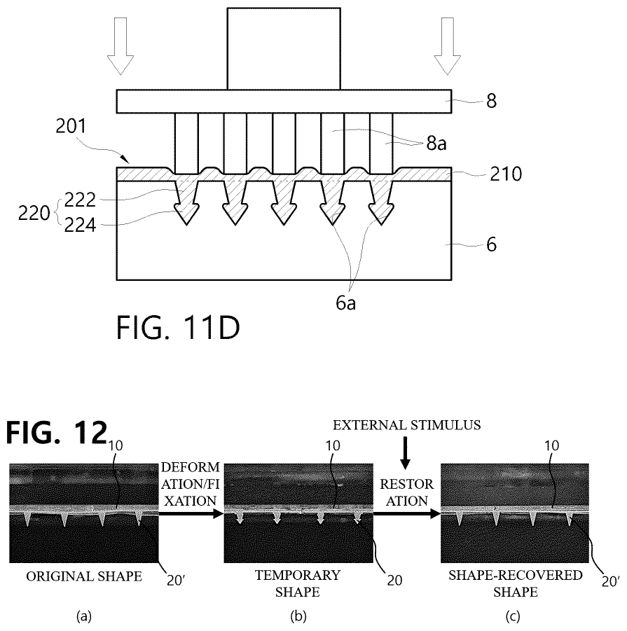

FIGS. 11A to 11D are diagrams for describing a method of manufacturing a microneedle structure according to a third embodiment of the present invention.

Referring to FIGS. 8, 11A, and 11B, the method of manufacturing a microneedle structure according to the third embodiment of the present invention includes disposing a shape-memory polymer 9″ on a mold 5′ for the second shape (S10, S20), disposing an intermediate member 4 on the upper side of the disposed shape-memory polymer 9″, and pressing the shape-memory polymer 9″ using the hot press plate 3 (S40). Accordingly, the shape-memory polymer 9″ is press-fitted into the intaglio 5a′ for the second shape.

In this case, the intaglio 5a′ for the second shape of the mold 5′ for the second shape may include an opening that is open upward and has a wide width, and a bottom surface made of a flat plane. The intaglio 5a′ for the second shape may be formed to a depth greater than or equal to the thickness of the finally formed support member 210 and the second shape 220′.

Next, a predetermined stimulus B is applied to the shape-memory polymer 9″ to cause a crosslinking reaction, thereby molding a portion of the shape-memory polymer 9″ into the second shape 220′ (S50). In this case, when the crosslinking reaction is a thermal crosslinking reaction, the predetermined stimulus B may be heat transfer by the hot press plate 3. On the other hand, when the crosslinking reaction is a photo-crosslinking reaction, the predetermined stimulus B may be composed of ultraviolet rays irradiated by a UV lamp. At this time, the upper portion of the shape-memory polymer 9″ may form the support member 210, and the lower portion of the shape-memory polymer 9″ may form the second shape 220′ of the microneedle.

Referring to FIGS. 8 and 11C, in the method of manufacturing a microneedle structure according to the third embodiment of the present invention, after the support member 210 and the second shape 220′ of the microneedle are formed on the upper side of the mold 6 for the first shape (S50), the temperature of the support member 210 and the second shape 220′ of the microneedle is adjusted to a variable temperature (S60).

In this case, a probe 8 may be disposed on the upper side of the support member 210. A plurality of micropillars 8a may be provided on a lower portion of the probe 8 to correspond to the plurality of intaglios 6a for the first shape formed on the upper surface of the mold 6 for the first shape.

Referring to FIG. 11D together, the micropillar 8a of the probe 8 presses the second shape 220′ of the microneedle into the intaglio 6a for the first shape (S70), and the microneedle is molded into the first shape 220 (S80). Thereafter, the support member 210 and the microneedles molded into the first shape 220 are cooled, and then maintained at a low temperature (S90), thereby fixing the shape of the microneedles to the first shape 220.

In this way, the method of manufacturing a microneedle structure according to the third embodiment of the present invention may manufacture the microneedle structure 201 according to the third embodiment of the present invention.

FIG. 12 is an image showing a microneedle structure array manufactured by the method of manufacturing a microneedle structure according to the embodiment of the present invention. Here, FIG. 12(a) is an image of microneedle structures molded into the second shape by any one step of the manufacturing method according to this embodiment, FIG. 12(b) is an image of the microneedle structures before an external stimulus is applied, and FIG. 12(c) is an image of the microneedle structures whose shape is deformed after the external stimulus is applied.

Referring to FIGS. 12A to 12C, it can be seen that the microneedle structure configured to be deformed from the first shape to the second shape in response to an external stimulus may be manufactured by the method of manufacturing a microneedle structure according to an embodiment of the present invention.

As described above, according to the microneedle and microneedle structure according to an embodiment of the present invention, and the method of manufacturing the same, the microneedles may be inserted into the skin in a state in which the microneedles have a shape having a high binding force with the skin, and may then be removed from the skin in a state in which the microneedles are deformed into a shape having a low binding force with the skin using the characteristics of the shape-memory polymer. Therefore, microneedles capable of being stably fixed to the skin and also easily removed from the skin when a drug is introduced into the body may be provided.

While one embodiment of the present invention has been described above, the spirit of the present invention is not limited to the embodiments proposed in this specification. Other embodiments may be easily suggested by adding, changing and removing components by those skilled in the art and will fall within the spirit and scope of the present invention.

1. A microneedle structure comprising microneedles to be inserted into the skin, comprising:
microneedles including a shape-memory polymer and formed to be deformed from a first shape to a second shape by a predetermined external stimulus according to the characteristics of the shape-memory polymer; and
a support member having the microneedles formed on one surface thereof.

2. The microneedle structure of claim 1, wherein the first shape includes:
a body portion having a cross section that becomes smaller toward one side thereof; and
a head portion extending from the one side of the body portion and having a cross section larger than that of the one side of the body portion.

3. The microneedle structure of claim 2, wherein the head portion has a cross-section that becomes smaller toward a front end thereof.

4. The microneedle structure of claim 1, wherein the second shape is a conical shape.

5. The microneedle structure of claim 1, wherein the second shape is formed flat on the one surface of the support member.

6. The microneedle structure of claim 1, wherein the microneedles are formed:
to be inserted into the skin while having the first shape,
to be deformed from the first shape to the second shape while being inserted into the skin, and
to be removed from the skin while having the second shape.

7. The microneedle structure of claim 1, wherein an outer shell layer is formed on outer surfaces of the microneedles, and
the outer shell layer includes a pharmaceutical composition.

8. The microneedle structure of claim 1, wherein an injection port is formed at the end of each of the microneedles,
an injection tube having one side connected to the injection port is formed in each of the microneedles, and
the microneedle structure further comprises a storage portion connected to the other side of the injection tube to accommodate the pharmaceutical composition.

9. The microneedle structure of claim 1, wherein the predetermined stimulus includes at least one of a temperature change or ultraviolet irradiation.

10. The microneedle structure of claim 1, wherein the shape-memory polymer includes at least one of a biodegradable shape-memory polymer or a biocompatible shape-memory polymer.

11. The microneedle structure of claim 10, wherein the biocompatible shape-memory polymer includes at least one of polycaprolactone with a crosslinked acrylated end group, network-structured polycaprolactone, a polycaprolactone blend with polyurethane, cellulose-grafted polycaprolactone, polycaprolactone-co-polysiloxane, multi-arm-structured polycaprolactone, multi-arm-structured poly(lactic acid), a poly(lactic acid) copolymer, and poly(lactic acid)-co-poly(ethylene glycol).

12. The microneedle structure of claim 11, wherein the predetermined stimulus includes a temperature change, and
the microneedles are gradually deformed as the temperature changes around the reference temperature, and has a first shape at a first temperature less than or equal to the reference temperature and a second shape at a second temperature higher than or equal to the reference temperature.

13. The microneedle structure of claim 12, wherein the reference temperature ranges from 28° C. to 42° C.

14. The microneedle structure of claim 1, wherein the microneedles are present in plural numbers, and
the plurality of microneedles are regularly arranged at regular intervals.

15. The microneedle structure of claim 14, wherein the plurality of microneedles include first and second microneedles, and
the first shape of the first microneedle is different from the first shape of the second microneedle.

16. A method of manufacturing a microneedle structure, wherein the microneedle structure includes microneedles including a shape-memory polymer and capable of being deformed from a first shape to a second shape by a predetermined external stimulus according to the characteristics of the shape-memory polymer; and a support member having the microneedles formed on one surface thereof, the method comprising:
molding microneedles having the second shape using the shape-memory polymer;
adjusting the temperature so that the shape of the microneedles having the second shape can be changed; and
molding the microneedles so that the microneedles whose temperature is adjusted have the first shape.

17. The method of claim 16, wherein the molding of the second shape includes:
applying the shape-memory polymer on one surface of a mold for the second shape on which an intaglio for the second shape corresponding to the second shape is formed; and
applying pressure so that a portion of the shape-memory polymer is introduced into the intaglio for the second shape.

18. The method of claim 17, wherein the molding of the second shape includes:
forming a support member by allowing the remainder other than the portion of the shape-memory polymer to spread on the one surface of the mold for the second shape centered on the intaglio for the second shape as pressure is applied to the shape-memory polymer.

19. The method of claim 17, wherein the molding of the second shape includes:
mixing a thermal crosslinking initiator with the shape-memory polymer; and
applying heat to the shape-memory polymer.

20. The method of claim 19, wherein the thermal crosslinking initiator includes at least one of potassium persulfate, ammonium persulfate, benzoyl peroxide, dilauroyl peroxide, dicumyl peroxide, hydrogen peroxide, and azobisisobutyronitrile.

21. The method of claim 17, wherein the molding of the second shape includes:
mixing a photo-crosslinking initiator with the shape-memory polymer; and
applying ultraviolet rays to the shape-memory polymer.

22. The method of claim 21, wherein the photo-crosslinking initiator includes at least one of Darocur, Irgacure, lithium phenyl-2,4,6-trimethylbenzoylphosphinate (LAP) having a phenyl phosphine structure, diphenyl(2,4,6-trimethylbenzoyl)phosphine (TPO), and ethyl(2,4,6-trimethylbenzoyl)phenylphosphinate (TPO-L).

23. The method of claim 16, wherein the molding of the first shape includes:
disposing the microneedles having the second shape in intaglios for the first shape formed on one surface of a mold for the first shape to correspond to the first shape; and
pressing the microneedles into the intaglios for the first shape.

24. The method of claim 23, comprising, after the molding of the first shape:
cooling the microneedles to a low temperature to fix the first shapes of the microneedles, and then maintaining the cooled state.

25. The method of claim 16, comprising, after the molding of the first shape:
attaching the microneedles having the first shape to the support member.

26. The method of claim 25, wherein the molding of the first shape includes:
disposing the microneedles having the second shape in the intaglios for the first shape formed on one surface of the mold for the first shape to correspond to the first shape;
applying an adhesive member to one side of each of the microneedles; and
pressing the microneedles into the intaglios for the first shape.
